(19) United States
(12) Patent Application Publication
(10) Pub. No.: US 2004/0261145 A1
(43) Pub. Date: Dec. 23, 2004

(54) Genetic transformation in plants using site-specific recombination and wide hybridization

(75) Inventors: Leszek Alexander Lyznik, Johnston, IA (US); Christopher L. Baszczynski, Urbandale, IA (US); Waclaw Orczyk, Blonie (PL)

Correspondence Address: PIONEER HI-BRED INTERNATIONAL INC., 7100 N.W. 62ND AVENUE, P.O. BOX 1000, JOHNSTON, IA, 50131, US

(73) Assignees: Pioneer Hi-Bred International, Inc.; Plant Breeding and Acclimatization Institute

(21) Appl. No.: 10/895,878

(22) Filed: Jul. 21, 2004

Related U.S. Application Data
(63) Continuation of application No. 09/850,492, filed on May 7, 2001, now abandoned.
(60) Provisional application No. 60/203,056, filed on May 8, 2000.

(51) Int. Cl. A01H 1/00; A01H 1/02; C12N 15/82; C12N 15/87

(52) U.S. Cl. 800/260; 800/266; 800/278

(57) ABSTRACT

The methods of the invention provide a means for targeting the insertion of a nucleotide sequence of interest to a specific chromosomal site within the genome of a plant cell. The invention provides a unique application of wide hybridization and site-specific recombination to bring together and recombine well defined chromosomal fragments. The invention provides novel methods to generate transgenic plant lines and new hybrid plant varieties.

### CROSS-REFERENCE TO RELATED APPLICATIONS

This application is a continuation of co-pending U.S. application Ser. No. 09/850,492 filed May 7, 2001, and claims the benefit of U.S. application Ser. No. 60/203,056 filed May 8, 2000, now abandoned, which are all herein incorporated in entirety by reference.

### FIELD OF THE INVENTION

The invention relates to the genetic modification of chromosomes. In particular, methods and compositions are provided for the control of gene integration and expression in plants using a site-specific recombination system.

### BACKGROUND OF THE INVENTION

Ongoing crop cultivar improvement is dependent on widening the genetic base of the crop plant through the introduction of new traits. Genetic modification techniques have been developed to enable one to insert exogenous nucleotide sequences of interest into the genome of a plant. For example, genetic enrichment of plants has been achieved through interspecific and intergeneric sexual hybridization or “wide crosses”. Such techniques have been successful in transferring a superior trait into a specific crop cultivar through the translocation of a chromosomal segment from a donor species that contains the genetic information encoding the desired trait. If the donor plant represents a primary or secondary gene pool species and has at least one genome in common with the recipient plant, recombination between homologous genomes can take place. Through several cycles of backcrossing and selection, the desired traits can be obtained. However, for successful transfers of this kind, chromosomes of the donor species and those of the acceptor must pair. Therefore, the relatedness of the donor and acceptor genome places severe limitations on the formulation of effective plant breeding programs (Jauhar et al. (1999) Genome 42:570-583). Furthermore, spontaneous translocation events are relatively infrequent, limiting the efficiency of this type of genetic enrichment procedure.

Methods for increasing the frequency of the recombination events between the donor and acceptor plants are known. For example, radiation can be used to induce chromosomal translocations. However, radiation results in the random breaking of chromosomes, and thus leads to unpredictable translocation events. For a chromosomal translocation event to be usable, i.e. agronomically desirable, the chromosomal translocation that occurs must be a compensating translocation. In other words, the resulting chromosomal translocation can not result in undesirable duplications and deficiencies in the plant's genome. Frequently, non-compensating translocations result in a reduction in plant vigor. Plants also often have reduced fertility, as gametes will have duplications and deficiencies.

Screening for compensating translocations is time consuming and tedious. Chromosomal rearrangements are most often revealed by aberrant phenotypes resulting from anomalous expression of the displaced genes. In other instances, identification of aberrant chromosome structures requires cytogenetic analysis, which makes the screening of large numbers difficult. Moreover, this method of inducing rearrangements lacks predictability and often causes additional mutations in the acceptor plants genome. Furthermore, many of these translocations also carry substantial portions of additional alien chromatin and require additional restructuring to make them suitable for use by plant breeders. Therefore, genetic modification techniques are needed that provide a means to direct well-defined chromosomal segments between two plant chromosomes.

Site specific recombination systems that rely on a single recombinase to direct the specific reciprocal exchange between two short identical DNA recombination sequences are known in the art. Such systems include Cre-lox, FLP-FRT, and R-RS. These systems consist of a specific recombination DNA sequence (lox, FRT, RS) and a recombinase (Cre, FLP, R) that is necessary and sufficient to induce cross-overs between two recombination sites.

Methods for the targeted integration of a DNA sequence of interest into a predetermined chromosomal location using a site-specific recombination system are described in detail in WO 99/25821; WO 99/25840; WO 99/25855; and WO 99/25854; all of which are herein incorporated by reference.

The methods of the present invention provide a unique application of wide hybridization and site-specific recombination systems to bring together and recombine well-defined chromosomal fragments.

### SUMMARY OF THE INVENTION

Compositions and methods are provided for targeting the insertion of a nucleotide sequence of interest to a specific chromosomal site within the genome of a plant cell. Specifically, the present invention provides a method of genomic DNA transfer between plant chromosomes using a site specific recombinase system. The methods of the invention comprise the generation of an acceptor and a donor plant. The acceptor plant has stably incorporated into its genome a target site comprising at least two non-identical recombination sites, while the donor plant has stably incorporated into its genome a transfer cassette. The transfer cassette of the donor plant comprises a nucleotide sequence of interest and at least two non-identical recombination sites that correspond to the sites found within the acceptor site.

Once the two plant lines are established, a genetically diverse male donor plant and a female acceptor plant are sexually crossed to one another. The newly formed zygote comprises genomes from both the donor and acceptor plants. The genetic diversity of the donor and acceptor plants results in the elimination of the donor chromosomes from the developing embryo. Prior to the chromosome elimination event, an appropriate site-specific recombinase is provided. The recombinase directs a recombination event between the recombination sites of the target site and the transfer cassette. The method of the invention results in the integration of the nucleotide sequences of interest into a predetermined genetic location of the acceptor plant genome. A haploid transgenic embryo comprising the nucleotide sequence of interest results. Subsequently, one can generate either a haploid or a diploid transgenic plant using techniques known in the art.

The combination of site-specific recombination utilizing donor and acceptor lines with DNA delivery via pollination can also be used to effect targeted gene insertion in the acceptor plant is not limited to genetically diverse lines. In this case, the resulting transgenic embryo is diploid and a diploid transgenic plant will result via normal development or using other techniques known in the art. The invention therefore provides novel methods for the establishment of transgenic plant lines and new hybrid plant varieties.

Compositions of the claimed invention comprise plants and plant seeds produced by the claimed method.

### DETAILED DESCRIPTION OF THE INVENTION

The methods of the invention provide a means for targeting the insertion of a nucleotide sequence of interest to a specific chromosomal site within the genome of a plant cell. The invention uses natural DNA delivery (i.e. fertilization of eggs) and a site-specific recombination system to direct the transfer of a DNA of interest between two plant chromosomes. Specifically, the methods of the invention comprise sexually crossing a genetically diverse male donor and female acceptor plant, wherein the donor and acceptor plant are from different species of either the same or different genera. The genetic diversity of the donor and acceptor plant is such as to result in the elimination of one set of parental chromosomes during embryo development. If the male donor and female acceptor are not genetically diverse, targeted insertion still occurs, but the male chromosomes are not eliminated during embryo development and a diploid embryo results.

The genome of the donor plant comprises at least one transfer cassette. The transfer cassette comprises a nucleotide sequence of interest flanked by non-identical recombination sites. The genome of the acceptor plant comprises a target site that is flanked by non-identical recombination sites that correspond to the sites found within the transfer cassette. The genomes of the donor plant and acceptor plant are brought together through fertilization methods. Prior to the elimination of the genome of the donor plant from the developing embryo, an appropriate recombinase is provided. The recombinase implements a double crossover recombination event between the recombination sites of the transfer cassette and the target site. The DNA of interest is thereby transferred from the chromosome of the donor plant into a predetermined chromosomal site (i.e., the target site) of the acceptor plant.

Following the recombination event and the elimination of the donor plant chromosomes, a haploid transgenic embryo or plant is produced. Subsequently, the haploid embryo is cultured in vitro using standard chromosomal doubling techniques to generate a diploid transgenic acceptor plant.

The method of the invention can be used for the directed DNA transfer between chromosomes of two plant species that are brought together as a result of sexual hybridization. The process can be used as a novel genetic transformation procedure for plants. Furthermore, the method can also be used to establish new hybrid plant varieties via the insertion of specific pre-determined chromosomal fragments into the genome of the acceptor plant.

The methods of the invention result in the transfer of a defined DNA fragment flanked by non-identical recombination sites into a predetermined chromosomal location. The natural process of fertilization serves merely as a DNA delivery system for the foreign DNA or chromosomal fragment. In the embodiments discussed below using genetically diverse donor and acceptor plants, any unspecified, heterologous DNA contamination from the genome of the donor plant will be minimized or eliminated shortly after fertilization. The methods provide a transgenic product containing a site-specific integration event of a nucleotide sequence of interest.

Establishment of Donor and Acceptor Plant Lines

The methods of the present invention require the establishment of two independent plant lines referred to herein as the “acceptor” plant and the “donor” plant. The acceptor and donor plants used in the methods of the present invention may be genetically diverse. By “genetically diverse” is intended the donor and acceptor plants are from different species of either the same or different genera. Hybridization of the genetically diverse acceptor and donor plants results in a haploid embryo. The donor and acceptor plants for use in the methods of the invention along with methods of the hybridization are described in more detail below.

Stably incorporated into the genome of the acceptor plant is a target site. By “target site” is intended a predetermined genomic location within the genome of the acceptor plant where a specific nucleotide sequence of interest is to be inserted. The target site of the acceptor plant is characterized by having recombination sites which correspond to the recombination sites in the transfer cassette. The target site may comprise only one recombination site, identical or dissimilar to the recombination sites of the transfer cassette. This would effect a single crossover integration of the transfer cassette into the acceptor target site. The target site may also be flanked by non-identical recombination sites which correspond to the non-identical recombination sites of the donor transfer cassette. This would effect a double reciprocal crossover exchange of donor transfer cassette into the acceptor target site. In this case, the target site comprises a first recombination site, one or more intervening nucleotide sequences, and a second recombination site, wherein the first and second recombination sites are non-identical. One or more intervening sequences may be present between the recombination sites of the target site. Intervening sequences of particular interest would include linkers, adapters, selectable markers, promoters and/or other sites that aid in vector construction or analysis. It is recognized that the acceptor plant may comprise multiple target sites; i.e., sets of non-identical recombination sites. In this manner, multiple manipulations of the target site in the acceptor plant are available. Additionally, as discussed in more detail below, the genome of the acceptor plant may also comprise an expression cassette comprising a nucleotide sequence encoding an appropriate recombinase.

The donor plant is characterized by the stable genomic integration of at least one DNA construct comprising a transfer cassette. As defined herein, the “transfer cassette” comprises a first recombination site, a nucleotide sequence of interest, and a second recombination site, wherein first and second recombination sites correspond to the recombination sites in the target site.

The recombination sites of the transfer cassette may be directly contiguous with the nucleotide sequence of interest or there may be one or more intervening sequences present between one or both ends of the DNA of interest and the recombination sites. Intervening sequences of particular interest would include linkers, adapters, selectable markers, promoters and/or other sites that aid in vector construction or analysis. Selectable markers of particular interest are described in more detail below. It is further recognized that the recombination sites can be contained within the nucleotide sequence of interest (i.e., such as within introns or untranslated regions).

The target site and transfer cassette are contained in their respective DNA constructs. It is recognized that the DNA construct can further comprise nucleotide sequences encoding selectable marker genes and/or promoter sequences that aid in selection of the recombination event (see Example 1). For example, a DNA construct can comprise a promoter located 5′ of and operably linked to the target site, such that the integration of a transfer cassette comprising a coding region into the target site results in expression of the coding sequences. For example, this embodiment would provide a method to select the transformed plants or plant cells if the coding region inserted comprises a selectable marker which, when integrated, is operably linked to the promoter 5′ of the target site.

Any transformation protocol may be used for the stable introduction of the DNA constructs comprising the target site and the transfer cassette into the genomes of the acceptor and donor plant. By “introducing” is intended presenting to the plant the nucleotide construct comprising the target site, transfer cassette, or recombinase, in such a manner that the construct gains access to the interior of a cell of the plant. The methods of the invention do not depend on a particular method for introducing a nucleotide construct into a plant, only that the DNA construct comprising the target site or the transfer cassette or the recombinase is stably incorporated into the genome. Methods for introducing nucleotide constructs into plants are known in the art and include, but are not limited to, stable transformation methods, transient transformation methods, and virus-mediated methods.

By “stable transformation” is intended that the nucleotide construct introduced into a plant integrates into the genome of the plant and is capable of being inherited by progeny thereof. Transformation protocols as well as protocols for introducing nucleotide sequences into plants may vary depending on the type of plant or plant cell, i.e., monocot or dicot, targeted for transformation. Suitable methods of introducing nucleotide sequences comprising the transfer cassette, target site, or appropriate recombinase into the donor or acceptor plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606), Agrobacterium-mediated transformation (Townsend et al., U.S. Pat. No. 5,563,055), direct gene transfer (Paszkowski et al. (1984) EMBO J. 3:2717-2722), and ballistic particle acceleration (see, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); and McCabe et al. (1988) Biotechnology 6:923-926). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); Finer and McMullen (1991) In Vitro Cell Dev. Biol. 27P:175-182 (soybean); Singh et al. (1998) Theor. Appi. Genet. 96:319-324(soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The cells from the donor and acceptor plants that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic imparted by the nucleotide sequence of interest and/or the genetic markers contained within the target site or transfer cassette. Two or more generations may be grown to ensure that expression of the desired phenotypic characteristic is stably maintained and inherited and then seeds harvested to ensure expression of the desired phenotypic characteristic has been achieved.

Site-Specific Recombination System

The methods of the invention employ a site-specific recombination system. By “site specific recombinase” is meant any enzyme that catalyzes conservative site-specific recombination between its corresponding recombination sites. For reviews of site-specific recombinases, see Sauer (1994) Current Opinion in Biotechnology 5:521-527; and Sadowski (1993) FASEB 7:760-767; the contents of which are incorporated herein by reference. The site-specific recombinase may be a naturally occurring recombinase or an active fragment or derivative thereof. Site-specific recombinases useful in the methods and compositions of the invention include recombinases from the integrase and resolvase families, derivatives thereof, and any other naturally occurring or recombinantly produced enzyme or derivative thereof, that catalyze conservative site-specific recombination between specified DNA sites. The integrase family of recombinases has over one hundred members and includes, for example, FLP, Cre, Int and R. For other members of the integrase family, see for example, Esposito et al. (1997) Nucleic Acid Research 25:3605-3614. Such site-specific recombination systems include, for example, the streptomycete bacteriophage phi C31 (Kuhstoss et al. (1991) J. Mol. Biol. 20:897-908); the SSV1 site-specific recombination system from Sulfolobus shibatae (Maskhelishvili et al. (1993) Mol. Gen. Genet. 237:334-342); and a retroviral integrase-based integration system (Tanaka et al. (1998) Gene 17:67-76). Preferably, the recombinase is one that does not require cofactors or a supercoiled substrate. Such recombinases include Cre, FLP, moFLP, and moCre.

The FLP recombinase is a protein that catalyzes a site-specific reaction that is involved in amplifying the copy number of the two micron plasmid of S. cerevisiae during DNA replication. The FLP recombinase catalyzes site-specific recombination between two FRT sites. The FLP protein has been cloned and expressed. See, for example, Cox (1993) Proc. Natl. Acad. Sci. U.S.A. 80:4223-4227. The FLP recombinase for use in the invention may be that derived from the genus Saccharomyces. One can also synthesize the recombinase using plant preferred codons for optimal expression in a plant of interest. A recombinant FLP enzyme containing maize preferred codons (moFLP) that catalyzes site-specific recombination events is known. See, for example, U.S. Pat. No. 5,929,301, herein incorporated by reference.

The bacteriophage recombinase Cre catalyzes site-specific recombination between two lox sites. The Cre recombinase is known in the art. See, for example, Guo et al. (1997) Nature 389:40-46; Abremski et al. (1984) J. Biol. Chem. 259:1509-1514; Chen et al. (1996) Somat. Cell Mol. Genet. 22:477-488; and Shaikh et al. (1977) J. Biol. Chem. 272:5695-5702, all of which are herein incorporated by reference. The Cre sequences may also be synthesized using plant preferred codons. Such sequences (moCre) are described in WO 99/25840, herein incorporated by reference.

It is further recognized that chimeric recombinases can be used in the methods of the present invention. By “chimeric recombinase” is intended a recombinant fusion protein which is capable of catalyzing site-specific recombination between recombination sites that originate from different recombination systems. That is, if the non-identical recombination sites utilized in the present invention comprise FRT and LoxP sites, a chimeric FLP/Cre recombinase will be needed or both recombinases may be separately provided. Methods for the production and use of such chimeric recombinases are described in WO 99/25840, herein incorporated by reference.

By “fragment” is intended a portion of the nucleotide sequence or a portion of the amino acid sequence and hence protein encoded thereby. Fragments of a nucleotide sequence encode a polypeptide which retains the biological activity of the recombinase and hence implements a recombination event. By “variant” protein is intended a protein derived from the native recombinase by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant recombinase enzymes encompassed by the present invention are biologically active, that is they continue to possess the desired biological activity of the native protein, that is, implement a recombination event between the appropriate recombination sites. Such variants may result from, for example, genetic polymorphism or from human manipulation. Biologically active variants of a native recombinase protein may have at least 75%, 80%, 85%, 90% to 95% or even 98% or more sequence identity to the amino acid sequence for the native protein as determined by sequence alignment programs described elsewhere herein using default parameters. A biologically active variant of a protein of the invention may differ from that protein by as few as 1 amino acid residues up to and including about 15 amino acid residues, such as 2, 3, 4, 5, 6, 7, 8, 9, 10, 11, 12, 13, 14 or more amino acid residues.

The recombinase used in the methods of the present invention may be altered in various ways including amino acid substitutions, deletions, truncations, and insertions. Methods for such manipulations are generally known in the art. For example, amino acid sequence variants of the recombinase protein can be prepared by mutations in the DNA. Methods for mutagenesis and nucleotide sequence alterations are well known in the art. See, for example, Kunkel (1985) Proc. Natl. Acad. Sci. USA 82:488492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar chemical properties, may be preferred.

The effect of the substitution, deletion, or insertion can be evaluated by routine screening assays known in the art. That is, the activity can be evaluated by the ability of the recombinase fragment or variant, upon introduction into cells containing appropriate FRT substrates, to catalyze site-specific recombination. For example, excision of a FRT flanked sequence that upon removal will activate an assayable marker gene.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Preferred, non-limiting examples of such mathematical algorithms are the algorithm of Myers and Miller (1988) CABIOS 4:11-17; the local homology algorithm of Smith et al. (1981) Adv. Appl. Math. 2:482; the homology alignment algorithm of Needleman and Wunsch (1970) J. Mol. Biol. 48:443-453; the search-for-similarity-method of Pearson and Lipman (1988) Proc. Natl. Acad. Sci. 85:2444-2448; the algorithm of Karlin and Altschul (1990) Proc. Natl. Acad. Sci. USA 87:2264, and the modification as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, California); the ALIGN program (Version 2.0) and GAP, BESTFIT, BLAST, FASTA, and TFASTA in the Wisconsin Genetics Software Package, Version 10 (available from Genetics Computer Group (GCG), 575 Science Drive, Madison, Wis., USA). Alignments using these programs can be performed using the default parameters. The CLUSTAL program is well described by Higgins et al. (1988) Gene 73:237-244; Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. The default parameters of a PAM120 weight residue table, a gap length penalty of 12, and a gap penalty of 4 can be used with the ALIGN program when comparing amino acid sequences. The BLAST programs of Altschul et al. (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. Several algorithms are available to search databases for more distantly related sequences, for example, Gapped BLAST (in BLAST 2.0) can be utilized as described in Altschul et al. (1997) Nucleic Acids Res. 25:3389-3402. Alternatively, PSI-BLAST (in BLAST 2.0) can be used to perform an iterated search that detects distant relationships between molecules. See Altschul et al. (1997) supra. When utilizing BLAST, Gapped BLAST, or PSI-BLAST, the default parameters of the respective programs (e.g., BLASTN for nucleotide sequences, BLASTX for proteins) can be used, and are publicly available, for example, on the NCBI website. Alignment may also be performed manually by inspection.

For purposes of the present invention, comparison of nucleotide or protein sequences for determination of percent sequence identity to the site-specific recombinase sequences is usually made using the GAP algorithm from the Wisconsin Genetics Software Package Version 10 under default parameters, or any equivalent program. By “equivalent program” is intended any sequence comparison program that, for any two sequences in question, generates a global alignment having identical nucleotide or amino acid residue matches and an identical percent sequence identity when compared to the corresponding alignment generated by the GAP algorithm.

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70% sequence identity, or at least 80%, or at least 90%, or at least 95%, compared to a reference sequence using one of the alignment programs described using standard parameters. One of skill in the art will recognize that these values can be appropriately adjusted to determine the corresponding identity of proteins encoded by two nucleotide sequences by taking into account codon degeneracy, amino acid similarity, reading frame positioning, and the like. Substantial identity of amino acid sequences for these purposes normally means sequence identity of at least 60%, or at least 70%, 80%, 90%, or 95%.

Another indication that nucleotide sequences are substantially identical is if two molecules hybridize to each other under stringent conditions. Generally, stringent conditions are selected to be about 5° C. lower than the thermal melting point (Tm) for the specific sequence at a defined ionic strength and pH. However, stringent conditions encompass temperatures in the range of about 1° C. to about 20° C. lower than the Tm, depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with an antibody directed to the polypeptide encoded by the second nucleic acid.

(e)(ii) The term “substantial identity” in the context of a peptide indicates that a peptide comprises a sequence with at least 70% sequence identity to a reference sequence, or at least 80%, 85%, 90% or 95% sequence identity to the reference sequence over a specified comparison window. Usually, alignment is conducted using the GAP global alignment algorithm of Needleman and Wunsch (1970) J. Mol. Biol. 48:443-453. An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides that are “substantially similar” share sequences as noted above except that residue positions that are not identical may differ by conservative amino acid changes.

The recombinase used in the methods of the present invention can be provided by any means known in the art. For example, the recombinase may be provided by stably incorporating into the genome of the acceptor plant an expression cassette comprising a nucleotide sequence encoding the site-specific recombinase operably linked to a promoter active in the plant. Any promoter, i.e. constitutive or inducible, that is capable of regulating expression in the plant may be used to express the appropriate site-specific recombinase. Specific examples of constitutive and inducible promoters useful in expressing the recombinase are provided below.

As described above, the target site and transfer cassette comprise recombination sites. It is recognized that the site-specific recombinase that is used in the invention will depend upon the recombination sites in the target site and the transfer cassette. That is, if FRT sites are utilized, the FLP recombinase will be needed. In the same manner, where lox sites are utilized, the Cre recombinase is required. If the non-identical recombination sites comprise both a FRT and a lox site, either a chimeric FLP/Cre recombinase or both FLP and Cre recombinases will be provided. Examples of recombination sites for use in the invention are known in the art and include FRT sites including, for example, the wild type FRT site (SEQ ID NO:1), and mutant FRT sites such as FRT5 (SEQ ID NO:2), FRT6 (SEQ ID NO:3) and FRT7 (SEQ ID NO:4). Recombination sites from the Cre/Lox site specific recombination system can also be used. Such recombination sites include, for example, wild type LoxP sites and mutant LoxP sites. An analysis of the recombination activity of mutant Lox sites is presented in Lee et al. (1998) Gene 216:55-65, herein incorporated by reference. Also, see for example, Schlake and Bode (1994) Biochemistry 33:12746-12751; Huang et al. (1991) Nucleic Acids Research 19:443-448; Paul D. Sadowski (1995) In Progress in Nucleic Acid Research and Molecular Biology Vol. 51, pp. 53-91; Michael M. Cox (1989) In Mobile DNA, Berg and Howe (eds) American Society of Microbiology, Washington D.C., pp. 116-670; Dixon et al. (1995) Mol. Microbiol. 18:449-458; Umlauf and Cox (1988) EMBO 7:1845-1852; Buchholz et al. (1996) Nucleic Acids Research 24:3118-3119; Kilby et al. (1993) Trends Genet. 9:413-421; Rossant and Geagy (1995) Nat. Med. 1: 592-594; Albert et al. (1995) The Plant J. 7:649-659; Bayley et al. (1992) Plant Mol. Biol. 18:353-361; Odell et al. (1990) Mol. Gen. Genet. 223:369-378; Dale and Ow (1991) Proc. Natl. Acad. Sci. USA 88:10558-10562; Qui et al. (1994) Proc. Natl. Acad. Sci. USA 91:1706-1710; Stuurman et al. (1996) Plant Mol. Biol. 32:901-913; Dale et al. (1990) Gene 91:79-85; and Albert et al. (1995) The Plant J. 7:649-659; all of which are herein incorporated by reference.

By “non-identical recombination sites” is intended that the flanking recombination sites are non-identical in sequence and that essentially will not recombine or recombination between the sites is minimal. That is, one flanking recombination site may be a FRT site where the second site may be mutant FRT site. Thus, suitable non-identical sites for use in the invention include those sites where the efficiency of recombination between the sites is low; for example, where the efficiency is less than about 30 to about 50%, preferably less than about 10 to about 30%, more preferably less than about 5 to about 10%, even more preferably less than about 1%. Accordingly, it is recognized that any suitable non-identical recombination sites may be utilized in the invention, including FRT and mutant FRT sites, FRT and lox sites, lox and mutant lox sites, and any other recombination sites known in the art.

As noted above, the recombination sites in the transfer cassette correspond to those in the target site of the acceptor plant. That is, if the target site of the acceptor plant contains flanking non-identical recombination sites of FRT and a mutant FRT, the transfer cassette of the donor plant will contain the same FRT and mutant FRT non-identical recombination sites.

Methods of Wide Hybridization

As discussed above, the present invention employs standard “wide hybridization” plant breeding techniques to bring together the genomic DNA of genetically diverse acceptor plants with the genomic DNA of a donor plant. The present invention encompasses sexual crosses between donor and acceptor plants of the same species, different species of the same genera (i.e. intrageneric crosses), between different genera (i.e. intergeneric) and even very high order wide crosses. “Wide hybridization” or “wide crosses” are defined herein as a method of sexually breeding individual plants at either the intrageneric or intergeneric levels. Methods for successful wide hybridization are known in the art, see for example, Fedak et al. (1999) Genome 42:584-591; Jauhar et al. (1999) Genome 42:570-583; Sharma et al. (1995) Euphytica 82:43-64; Laurie et al. (1989) Genome 32:953-61; Matzak et al. (1994) Plant Breeding 113:129; Inagaki et al. (1995) Breeding Science 45:157-161; Zhang et al. (1996) Euphytica 90:315-324; and Levfebvre et al. (1996) Theor App Genet 93:1267-1273; all of which are herein incorporated by reference.

As used herein “sexually crossing” encompasses any means by which two haploid gametes are brought together resulting in a successful fertilization event and the production of a zygote. By “gamete” is intended a specialized haploid cell, either a sperm or an egg, serving for sexual reproduction. By “zygote” is intended a diploid cell produced by fusion of a male and female gamete (i.e. a fertilized egg). The resulting “hybrid” zygote contains chromosomes from both the acceptor and donor plant. The zygote then undergoes a series of mitotic divisions to form an embryo.

Depending on the relatedness or genetic diversity of the parental genomes, wide crosses can result in a karyotypically stable or unstable embryo (Jauher et al. (1999) Genome 42:570-583). In the some embodiments of the present invention, wide crosses are performed which result in karyotypically unstable embryos. This type of wide cross is performed between parental plants having a low degree of genomic relatedness and results in the elimination of the male chromosomes from the developing embryo. Elimination of the unstable chromosomes may occur at the zygotic stage or following the first mitotic division. A haploid embryo comprising the genome of the acceptor plant results.

The targeted genomic insertion of a DNA sequence of interest using a site-specific recombination method can be achieved using a genetically diverse acceptor plant and donor plant which when crossed form a karyotypically unstable embryo. More specifically, a female acceptor plant, having stably incorporated into its genome a DNA construct comprising the target site and an expression cassette comprising an appropriate recombinase, is crossed to a male donor plant. The genome of the male donor plant comprises the transfer cassette with the nucleotide sequence of interest. Prior to the elimination of the donor chromosomes from the newly formed hybrid zygote, site-specific recombination occurs between the target sites of the acceptor plant genome and the transfer cassette of the donor plant genome. Subsequently, the chromosomes of the male donor plant are eliminated from the embryo. Depending on the timing of chromosomal elimination, a transgenic haploid embryo or a transgenic haploid zygote is formed. If the donor plant and acceptor plant are not genetically diverse, cross hybridization results in site-specific recombination of the nucleotide sequence of interest from the transfer cassette to the target site of the acceptor plant, forming a karyotypically stable embryo. The chromosomes from the male donor plant will be retained and the embryo will develop via the normal post-fertilization pathway. As defined herein, the “transgenic” plant comprises a stably integrated DNA sequence of interest in a predetermined genomic location of the acceptor plant chromosome.

After wide hybridization, a haploid transgenic embryo will be produced. Using methods known in the art, this embryo can be cultured to produce a transgenic haploid plant. Using further methods known in the art, this transgenic haploid embryo can be induced to undergo chromosomal doubling, from which can be generated a diploid transgenic plant. In vitro techniques that promote chromosomal doubling are known in the art. For instance, anti-microtubule agents such as APM, pronamide, and colchicine can be used to induce chromosome doubling. See, for example, Wan et al. (1995) Plant Breeding 114:253-255 and Lefebvre et al. (1996) Theoretical and Applied Genetics 93:1267-1273, both of which are herein incorporated by reference. The transgenic diploid embryo can then be grown into a transgenic diploid plant. The transgenic diploid plants can be used in subsequent self fertilization crosses or outcrosses to ensure the expression of the desired phenotypic characteristics and to produce seed.

In some embodiments, the loss of chromosomal content from the acceptor plant is minimized, and in further embodiments, the loss of chromosomal content from the genome of the acceptor plant is completely prevented. Furthermore, in certain embodiments, the resulting transgenic embryo will contain a minimal amount of heterologous DNA from the donor plant. In further embodiments, the transgenic embryo does not contain any contaminating heterologus DNA from the donor plant. It is recognized that in some wide crosses the elimination of the donor chromosomes from the embryo may not always be complete. In this instance, a stable partial hybrid embryo results. Such embryos have a complete haploid set of chromosomes from the acceptor plant and one or more chromosomes from the donor plant. Such stable partial hybrids have been obtained between oat x maize crosses. See for example, Riera-Lizaraza (1996) Theor Appl Genetics 93:123-135, herein incorporated by reference. The method of the present invention therefore provides methods to establish new hybrid plant varieties. Methods to determine if heterologous DNA from the donor plant chromosome is present in the transgenic acceptor embryo or plant are known in the art. Such methods include chromosome counting, genomic in situ hybridizations, genomic DNA restriction digestions, and southern transfer.

Pre- and post-fertilization barriers may hamper successful sexual wide hybridizations. Methods of overcoming these barriers are reviewed by Sharma et al. (1995) Euphytica 82:43-64, herein incorporated by reference. Factors directly related to vigor of plants, such as developmental stage with florets, application of growth regulators, intra-ovarian fertilization, and in vitro culture of rescued embryos can be modified to increase the overall efficiency of the wide hybridization process. For instance, post-pollination application of growth regulators, such as, gibberellic acid, naphthalene acetic acid, kinetin, or 2,4-D singly or as a mixture are known to facilitate embryo growth. Examples of such growth regulator combinations include, 2,4-D 20 mgl⁻¹, GA₃ 75 mgl⁻¹ and 2,4-D 18 mgl⁻¹, Dicamba, 9 mgl⁻¹, BA 2 mgl⁻¹ (Giura, A (1997) In Current Topics in Plant Cytogenetics Related to Plant Improvement; Inagaki et al. (1995) Breeding Science 45:21-24; O'Donoughue et al. (1994) Theor. Appl. Genet. 89:559-566; and Wedzong et al. (1998) Plant Breeding 117:211-215). Alternatively, a single treatment with 2,4-D or Dicamba two to four days after pollination sufficiently stimulated embryos to be ready for excision and in vitro culture 15-18 days later (Matzk, F and Mahn, A (1994) Plant Breeding 113:125-129; and Inagaki et al. (1995) Breeding Science 45:157-161).

Isolated embryos are cultured in vitro. Medias used in the methods of culturing are known in the art and include, but are not limited to 190-2 (Zhuang et al. (1983) In Cell and Tissue Culture Techniques for Cereal Crop Improvement 431, Hu and Vega, Eds., Science Press) or MS supplemented with IAA 0.1 mgl⁻¹, kinetin 1 mgl⁻¹, sucrose 601 gl⁻¹ (Zhang et al. (1996) Euphytica 90:315-324). Both of these references are herein incorporated by reference.

It is recognized that any plant may be stably transformed with a DNA construct comprising a transfer cassette or a target site and used as a donor or acceptor plant in the methods of the present invention. Such plants include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats (Avena spp.), barley (Hordeum spp.), vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesil); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecypans nootkatensis).

In certain embodiments, acceptor and donor plants used in the methods of the present invention may be crop plants (for example, corn, alfalfa, sunflower, Brassica, soybean, cotton, safflower, peanut, sorghum, wheat, millet, tobacco, etc.), particularly corn and soybean plants, or plants from the family Poaceae that include, but are not limited to, members of the genera, Zea (maize), Triticum (wheat), Hordeum (barley), Avena (oats), Secale (rye), Sorghum, Pennisetum, Agropyron, Aegilops, Haynaldia, Lophopyrcum and Thinopyrum. Any species from these various genera may be used as an acceptor or donor plant line in the methods of the invention. Of particular interest are donor plants lines from Zea and acceptor plant lines from Zea or Triticum.

Wide crosses that result in karyotypically unstable hybrid embryos are known in the art. For a review see, Sharma et al. (1995) Euphytica 82:43-64, herein incorporated by reference. Such crosses include, but are not limited to, hexaploid wheat and H. bulbosum (Barclay (1975) Nature 256:410-411; and Sitch (1984) Ph.D. thesis, University of Cambridge, Cambridge, U.K.), or sorghum (Laurie et al. (1988) Plant Breed 100:73-82, or pearl millet (Laurie et al. (1989) Genome 32:963-61); between tetraploid wheat and maize (O'Donoughue et al. (1988) Proceedings of the 7^{th} International Wheat Genetics Symposium), or pearl millet (Laurie et al. (1989) Genome 32:963-61); between barley and maize (Laurie et al. (1988) New Chromosome Conf. Proc. 3^{rd}167-177); between barley H. bulbosum crosses (Subrahmanyam et al. (1973) Chromosome 42:111-125; Bennett et al. (1976) Chromosoma 54:175-200; Finch et al. (1983) New Chromosome Conf. Proc. 2^{nd}147-154); and between maize and oat (Riera-Lizarazu et al. (1996) Theor Appl Genet 93:123-135).

It is well recognized in the art that the genetic variation within a plant species can influence the success of the wide cross by facilitating fertilization or seed development until embryo rescue is possible. For instance, the crossability inhibiting genes (Kr genes), pose a major obstacle to hybridizing wheat with related genera. However, high crossability genes, such as kr1, kr2, and kr3, in wheat cultivars like Chinese Spring (CS) and kr4 in landraces of wheat have been shown to facilitate crossability of wheat with species of other genera. See, for example, Miller et al. (1983) Canad. J. Genet. Cytol. 25;634-641; Lou et al. (1993) Euphytica 67:1-8; and Jauhar et al. (1999) Genome 42:570-583. Accordingly, it is well within skill in the art to select plant cultivars to be used in the wide cross of the present invention that have genetic backgrounds that improve, for example, the frequency of successful fertilization and/or the overall survival of the embryo.

Nucleotide Sequence of Interest and Methods of Expression

The methods of the present invention provide a method for the targeted insertion of a DNA sequence of interest into the genome of a plant. The DNA sequence of interest may impart various changes in phenotype in the transgenic plant produced by the targeted insertion including, but not limited to, modification of the fatty acid composition in the plant, altering the amino acid content of the plant, altering the plant's pathogen defense mechanism, and the like. These results can be achieved by providing expression of heterologous products or increased expression of endogenous products in plants.

Nucleotide sequences of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases, and those involved in housekeeping, such as heat shock proteins. More specific categories of transgenes, for example, include sequences encoding important traits for agronomics, insect resistance, disease resistance, herbicide resistance, sterility, grain characteristics, and commercial products. Nucleotide sequences of interest include, generally, those involved in oil, starch, carbohydrate, protein, or nutrient metabolism as well as those affecting kernel size, sucrose loading, and the like.

Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802, and 5,990,389 herein incorporated by reference. Another example is lysine and/or sulfur rich seed protein encoded by the soybean 2S albumin described in U.S. Pat. No. 5,850,016, and the chymotrypsin inhibitor from barley, described in Williamson et al. (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, which is described in WO 98/20133, the disclosure of which is herein incorporated by reference. Other proteins include methionine-rich plant proteins such as from sunflower seed (Lilley et al. (1989) Proceedings of the World Congress on Vegetable Protein Utilization in Human Foods and Animal Feedstuffs, ed. Applewhite (American Oil Chemists Society, Champaign, Ill.), pp. 497-502; herein incorporated by reference); corn (Pedersen et al. (1986) J. Biol. Chem. 261:6279; Kirihara et al. (1988) Gene 71:359; both of which are herein incorporated by reference); and rice (Musumura et al. (1989) Plant Mol. Biol. 12:123, herein incorporated by reference). Other agronomically important genes encode latex, Floury 2, growth factors, seed storage factors, and transcription factors.

Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,737,514; 5,723,756; 5,593,881; and Geiseretal. (1986) Gene 48:109); lectins (Van Damme et al. (1994) Plant Mol. Biol. 24:825); and the like.

Genes encoding disease resistance traits include detoxification genes, such as against fumonosin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene), or other such genes known in the art. The bar gene encodes resistance to the herbicide basta, the nptll gene encodes resistance to the antibiotics kanamycin and geneticin, and the ALS-gene mutants encode resistance to the herbicide chlorsulfuron.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

The quality of grain is reflected in traits such as levels and types of oils, saturated and unsaturated, quality and quantity of essential amino acids, and levels of cellulose. In corn, modified hordothionin proteins are described in U.S. Pat. Nos. 5,703,049, 5,885,801, 5,885,802, and 5,990,389 herein incorporated by reference.

Commercial traits can also be encoded on a gene or genes that could, for example increase starch for ethanol production, or provide expression of proteins. Another important commercial use of transformed plants is the production of polymers and bioplastics such as described in U.S. Pat. No. 5,602,321. Genes such as β-Ketothiolase, PHBase (polyhydroxyburyrate synthase), and acetoacetyl-CoA reductase (see Schubert et al. (1988) J. Bacteriol. 170:5837-5847) facilitate expression of polyhyroxyalkanoates (PHAs).

Exogenous products include plant enzymes and products as well as those from other sources including prokaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content.

Furthermore, it is recognized that the nucleotide sequence of interest may also comprise antisense sequences complementary to at least a portion of the messenger RNA (mRNA) for a targeted gene sequence of interest. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, 80%, or 85% sequence identity to the corresponding antisensed sequences may be used. Furthermore, portions of the antisense nucleotides may be used to disrupt the expression of the target gene. Generally, sequences of at least 50 nucleotides, 100 nucleotides, 200 nucleotides, or greater may be used.

In addition, the nucleotide sequences of interest may also be used in the sense orientation to suppress the expression of endogenous genes in plants. Methods for suppressing gene expression in plants using nucleotide sequences in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, generally greater than about 65% sequence identity, about 85% sequence identity, or greater than about 95% sequence identity. See U.S. Pat. Nos. 5,283,184 and 5,034,323; herein incorporated by reference.

The nucleotide sequences encoding the DNA sequences of interest are provided in expression cassettes for insertion into the transfer cassette. In addition, in specific embodiments of the present invention, the nucleotide sequence encoding an appropriate recombinase is also contained in an expression cassette. The cassette will include 5′ and 3′ regulatory sequences operably linked to the DNA sequence of interest. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. The cassette may additionally contain at least one additional gene to be cotransformed into the organism. Alternatively, the additional gene(s) can be provided on multiple expression cassettes.

Such an expression cassette is provided with a plurality of restriction sites for insertion of the DNA sequence of interest to be under the transcriptional regulation of the regulatory regions. The expression cassette may additionally contain selectable marker genes.

The expression cassette will include in the 5′-3′ direction of transcription, a transcriptional and translational initiation region, a DNA sequence of interest, and a transcriptional and translational termination region functional in plants. In other embodiments, the expression cassette comprises a nucleotide sequence of interest 5′ to a translational termination region functional in plants. In this embodiment, the target site comprises a promoter 5′ to the recombination sites, thereby, upon recombination, the nucleotide sequence of interest is operably linked to the promoter sequence.

The transcriptional initiation region, the promoter, may be native, analogous, foreign, or heterologous to the plant host or to the DNA sequence of interest. Additionally, the promoter may be the natural sequence or alternatively a synthetic sequence. By “foreign” is intended that the transcriptional initiation region is not found in the native plant into which the transcriptional initiation region is introduced. Such constructs would change expression levels of DNA sequence of interest in the plant or plant cell. Thus, the phenotype of the plant or plant cell is altered.

The termination region may be native with the transcriptional initiation region, may be native with the operably linked DNA sequence of interest, or may be derived from another source. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

Where appropriate, the nucleotide sequence of interest or the recombinase may be optimized for increased expression in the transformed plant. That is, the genes can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, N. Y.), pp. 237-256); and maize chlorotic mottle virus leader (MCMV) (Lommel et al. (1991) Virology 81:382-385). See also, Della-Cioppa et al. (1987) Plant Physiol. 84:965-968. Other methods or sequences known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. For instance, the recombinase and/or the nucleotide sequence of interest can be combined with constitutive, tissue-preferred, or other promoters for expression in plants.

Examples of constitutive promoters include, for example, the core promoter of the Rsyn7 (WO 99/43838); the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; and 5,608,142.

In addition, chemical-regulated promoters can be used to modulate the expression of a gene in a plant through the application of an exogenous chemical regulator. Depending upon the objective, the promoter may be a chemical-inducible promoter, where application of the chemical induces gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-la promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Generally, the expression cassette will comprise a selectable marker gene for the selection of transformed cells. Selectable marker genes are utilized for the selection of transformed cells or tissues. Marker genes include genes encoding antibiotic resistance, such as those encoding neomycin phosphotransferase 11 (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). See generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); and Gill et al. (1988) Nature 334:721-724. Such disclosures are herein incorporated by reference.

The above list of selectable marker genes is not meant to be limiting. Any selectable marker gene can be used in the present invention.

Other Embodiments of Site-Specific Recombination System

It is recognized that many variations of the site-specific recombination system are known in the art and may be used in combination with the DNA delivery system described above for the transfer a nucleotide sequence of interest into a predetermined chromosomal location. For example, the target sites of the acceptor plant can be constructed to have multiple non-identical recombination sites. Thus, multiple genes or nucleotide sequences can be stacked or ordered at a precise location in the genome of the acceptor plant. Likewise, once a target site has been established within the genome, additional recombination sites may be introduced by incorporating such sites within the nucleotide sequence of the transfer cassette. Thus, once a target site has been established, it is possible to subsequently add sites, or alter sites through recombination. Such methods are described in detail in WO 99/25821, herein incorporated by reference.

For instance, the genome of the acceptor plant can comprise a first target site comprising at least three non-identical recombination sites, wherein the first and second sites are in near proximity, and herein referred to as the first retargeting site. The second and third sites are in near proximity, and referred to as the second retargeting site. As used herein, the term “near proximity” means that the recombination sites are located at distance relative to each other such that the appropriate recombinase can efficiently catalyze a site-specific recombination event. The genome of the donor plant comprises at least one DNA construct containing a transfer cassette with a first recombination site, a nucleotide sequence of interest, and a second recombination site. The first and second sites are non-identical and correspond to the recombination sites of the first retargeting site. The donor and acceptor plants are sexually crossed and an appropriate recombinase is provided that implements recombination at the non-identical recombination sites. A transgenic plant is generated as a result of this cross and site-specific recombination. The steps are repeated using a donor plant containing within its genome a transfer cassette comprising the recombination sites of the second retargeting site and a second nucleotide sequence of interest. It is recognized that the target site can contain more than two retargeting sites, allowing for multiple nucleotide sequences of interest to be “stacked” in a predetermined position of the genome of the acceptor plant.

In another variation of the present invention a plurality of copies of the nucleotide sequence of interest is provided to the embryo. This approach may be accomplished by the incorporation of an autosomal self-replicating unit into the transfer cassette. For example, a viral replicon may be inserted in the transfer cassette. Such a method is described in detail in WO 99/25855. In this embodiment, the transfer cassette comprises both a viral replicon and the nucleotide sequence of interest. Specifically, the transfer cassette, which is stably incorporated into the genome of the donor plant, comprises in a 5′ to 3′ or 3′ to 5′ orientation: a first recombination site, a viral replicon, a second recombination site, the DNA sequence of interest, and a third recombination site. The first and third recombination site of this transfer cassette are directly repeated and identical with respect to each, and the second recombination site is non-identical to the first and third target site.

By “directly repeated” is meant that the target sites that flank the viral DNA are arranged in the same orientation, so that recombination between these sites results in excision, rather than inversion, of the viral DNA.

The acceptor and donor plants are sexually crossed as discussed above. When an appropriate recombinase is provided, the transfer cassette flanked by the directly repeated target sites is excised from the genome of the donor plant, producing a viable viral replicon containing the nucleotide sequence of interest. Replication of this viral replicon will result in a high number of copies of the replicon and also prolong the availability of the donor transfer cassette within the cell. The inclusion of the non-identical recombination site between the viral replicon and the DNA of interest allows integration of the DNA of interest into the target site flanked by the corresponding non-identical recombination sites of the acceptor plant. In this embodiment, the acceptor plant genome comprises an expression cassette containing the site-specific recombinase.

By “viral replicon” is meant double-stranded DNA from a virus having a double stranded DNA genome or replication intermediate. The excised viral DNA is capable of acting as a replicon or replication intermediate, either independently, or with factors supplied in trans. The viral DNA may or may not encode infectious viral particles and furthermore may contain insertions, deletions, substitutions, rearrangements or other modifications. The viral DNA may contain heterologous DNA. In this case, heterologous DNA refers to any non-viral DNA or DNA from a different virus. For example, the heterologous DNA may comprise an expression cassette for a protein or RNA of interest.

Viral replicons suitable for use in the methods and compositions of the invention include those of viruses having a circular DNA genome or replication intermediate, such as: Abuitilon mosaic virus (AbMV), African cassava mosaic virus (ACMV), banana streak virus (BSV), bean dwarf mosaic (BDMV), bean golden mosaic virus (BGMV), beet curly top virus (BCTV), beet western yellow virus (BWYV) and other luteoviruses, cassava latent virus (CLV), carnation etched virus (CERV), cauliflower mosaic virus (CaMV), chloris striate mosaic (CSMV), commelina yellow mottle virus (CoYMV), cucumber mosaic virus (CMV), dahlia mosaic virus (DaMV), digitaria streak virus (DSV), figwort mosaic virus (FMV), hop stunt viroid (HSV), maize streak virus (MSV), mirabilias mosaic virus (MMV), miscanthus streak virus (MiSV), potato stunt tuber virus (PSTV), panicum streak virus (PSV), potato yellow mosaic virus (PYMV), rice tungro bacilliform virus (RTBV), soybean chlorotic mottle virus (SoyCMV), squash leaf curl virus (SqLCV), strawberry vein banding virus (SVBV), sugarcane streak virus (SSV), thistle mottle virus (ThMV), tobacco mosaic virus (TMV), tomato golden mosaic virus (TGMV), tomato mottle virus (TMoV), tobacco ringspot virus (TobRV), tobacco yellow dwarf virus (TobYDV), tomato leaf curl virus (TLCV), tomato yellow leaf curl virus (TYLCV), tomato yellow leaf curl virus-Thailand (TYLCV-t) and wheat dwarf virus (WDV) and derivatives thereof. In some embodiments, the viral replicon may be from MSV, WDV, TGMV or TMV.

It is further recognized that the insertion of a nucleotide sequence of interest into the genome of the acceptor plant can occur via a single cross over event. For instance, the transfer cassette can comprise a first recombination site, an autosomal self-replicating unit, a DNA sequence of interest, and a second recombination site. The first and second recombination sites of the transfer cassette are identical and direct repeats. The target site of the acceptor plant comprises a single recombination site that is “dissimilar” to the recombination sites of the transfer cassette. By “dissimilar” recombination sites is intended that the recombination sites are not identical to one another but remain able to undergo a recombination event with one another. The dissimilar recombination sites are designed such that integrative recombination events are favored over the excision reaction. Such dissimilar recombination sites are known in the art. For example, Albert et al. introduced nucleotide changes into the left 13 bp element (LE mutant lox site) or the right 13 bp element (RE mutant lox site) of the lox site. Recombination between the LE mutant lox site and the RE mutant lox site produces the wild-type loxP site and a LE+RE mutant site that is poorly recognized by the recombinase Cre, resulting in a stable integration event (Albert et al. (1995) Plant J. 7:649-659). See also, for example, Araki et al. (1997) Nucleic Acid Research 25:868-872.

As discussed above, the acceptor plant and donor plant comprising the target site and the transfer cassette are crossed. When an appropriate recombinase is provided, a recombination event between the identical recombination sites of the transfer cassette occurs. This event results in excision of the autosomal self-replicating unit from the genome of the donor plant. Replication of the self-replicating unit results in a high copy number of the vector in the acceptor plant cell and prolongs the availability of the donor transfer cassette in the cell. A second recombination event between the dissimilar recombination sites of the target site and transfer cassette allows the stable integration of the self-replicating unit and the DNA sequence of interest at the target site of the acceptor plant.

The following examples are offered by way of illustration and not by way of limitation.

### EXPERIMENTAL

### Example 1

### Generation of Transformation Vectors Comprising the Target Site and Acceptor Sites.

DNA constructs are generated comprising either the transfer cassette or the target site and are used in plant transformations to establish the donor and acceptor plant lines, respectively. The target site and the transfer cassette contained within these vectors comprise a set of genetic markers convenient for kinetic analysis of recombination events and a set of markers allowing the selection of chromosomal-exchange events. This example describes the use of FRT recombination sites and a FLP recombinase, but any site-specific recombination system can be used in the present invention.

The transfer cassette comprises a recombination site, for example FRT, a marker gene expression cassette, such as gusA or GFP, a promoter active in the plant, such as the maize ubiquitin or CaMV 35S promoter, and a second recombination site, such as mutant FRT (FRT'). For example, as described above the transfer cassette can comprise FRT::promoter+GUS::ubiquitin promoter::FRT'. Further, an expression cassette comprising a nucleotide sequence of interest may be inserted upstream of the promoter. For example, the transfer cassette can comprise FRT::promoter+GUS::nucleotide sequence of interest::ubiquitin promoter::FRT'.

The DNA construct containing the target site comprises the non-identical recombination sites used in the transfer cassette FRTand FRT'. Immediately 3′ to the second recombination site of the target site is a promoterless marker gene (bar). The target site described above comprises FRT::FRT'::bar. Recombination between the transfer cassette and the target site places a promoter, in this example ubiquitin promoter, upstream of the bar gene which results in the expression of bar.

Standard molecular biology and cloning techniques are used to generate DNA constructs comprising the target site and transfer cassette and the associated marker genes and promoters. The DNA constructs are then inserted into the desired transformation vectors. See, for example, Sambrook et al., Molecular Cloning: A Laboratory Manual 2^{nd} ed. (1989) Cold Spring Harbor Laboratory Press, herein incorporated by reference.

Standard molecular biology techniques are also used to generate a transformation vector comprising a nucleotide sequence encoding the FLP recombinase operably linked to the maize ubiquitin promoter.

### Example 2

### Generation of Donor and Acceptor Plants

A. Transformation and Regeneration of Acceptor and Donor Plants by Agrobacterium-Mediated Transformation

It is noted that donor and acceptor plants can be established by any method of transformation. For example, donor and acceptor plant lines can be established via Agrobacterium mediated infection or particle bombardment. If transformation is performed using Agrobacterium mediated transformation methods the transfer cassette and target sites will be inserted into the T-DNA of an Agrobacterium binary vector as described by Bevin et al. (1984) Nucleic Acids Research 12:8711-8721 herein incorporated by reference.

For Agrobacterium-mediated transformation of maize with a DNA construct comprising a transfer cassette, generally the method of Zhao is employed as contained in U.S. Pat. No. 5,981,840, the contents of which are hereby incorporated by reference. Briefly, immature embryos are isolated from maize and the embryos contacted with a suspension of Agrobacterium, where the bacteria are capable of transferring the target site or the transfer cassette into at least one cell of at least one of the immature embryos (step 1: the infection step). In this step the immature embryos are immersed in an Agrobacterium suspension for the initiation of inoculation. The embryos are co-cultured for a time with the Agrobacterium (step 2: the co-cultivation step). The immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). The immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). The immature embryos are cultured on solid medium with a selective agent resulting in the selective growth of transformed cells. The callus is then regenerated into plants (step 5: the regeneration step), and calli grown on selective medium are cultured on solid medium to regenerate the plants. The acceptor plant will be monitored for phenotypic traits associated with both the site specific recombinase and the target site.

Agrobacterium-mediated transformation can also be used to stably introduce into the genome of the wheat acceptor plant an expression cassette containing the site specific recombinase and a DNA construct comprising a target site. See, for example, Cheng et al. (1997) Plant Physiology 115:971-980. The donor plants will be monitored for the phenotypic trait associated with the marker gene for the transfer cassette.

B. Transformation and Regeneration of Acceptor and Donor Plants By Bombardment

Maize

Immature maize embryos from greenhouse donor plants are bombarded with a plasmid containing either a donor transfer cassette or a acceptor target site DNA construct as described in Example 1. The plasmid may also contains the selectable marker gene PAT (Wohlleben et al. (1988) Gene 70:25-37) that confers resistance to the herbicide Bialaphos. Transformation is performed as follows. Media recipes follow below.

Preparation of Target Tissue

The ears are surface sterilized in 30% Chlorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5-cm target zone in preparation for bombardment.

Preparation of DNA

The plasmid DNA describe above is precipitated onto 1.1 μm (average diameter) tungsten pellets using a CaCl₂ precipitation procedure as follows:

100 μl prepared tungsten particles in water

10 μl (1 μg) DNA in TrisEDTA buffer (1 μg DNA total)

100 μl 2.5 M CaC1₂

10 μl 0.1 M spermidine

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, and the particles are washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

Transformation and Regeneration

The sample plates are bombarded at level #4 in a DuPont PDS 1000/He gun. All samples receive a single shot at 650 PSI, with a total of ten aliquots taken from each tube of prepared particles/DNA.

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to Classic 600 pots (1.6 gallon) and grown to maturity. The donor plant will be monitored for the phenotypic trait associated with the marker gene for the transfer cassette.

Bombardment medium (560Y) comprises 4.0 g/l N6 basal salts (SIGMA C-1416), 1.0 ml/l Eriksson's Vitamin Mix (1000×SIGMA-1511), 0.5 mg/l thiamine HCl, 120.0 g/l sucrose, 1.0 mg/l 2,4-D, and 2.88 g/l L-proline (brought to volume with D-I H₂O following adjustment to pH 5.8 with KOH); 2.0 g/l Gelrite (added after bringing to volume with D-I H₂O); and 8.5 mg/l silver nitrate (added after sterilizing the medium and cooling to room temperature). Selection medium (560R) comprises 4.0 g/l N6basal salts (SIGMA C-1416), 1.0 ml/l Eriksson's Vitamin Mix (1000×SIGMA-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite (added after bringing to volume with D-I H₂O); and 0.85 mg/l silver nitrate and 3.0 mg/l bialaphos(both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/l myo-inositol, and 40.0 g/l sucrose (brought to volume with polished D-I H₂O after adjusting pH to 5.6); and 6 g/l bacto-agar (added after bringing to volume with polished D-I H₂O), sterilized and cooled to 60° C.

Wheat

Preparation of Target Tissue trays for vernalisation at 6° C. for eight weeks. Vernalized seedlings are transferred in 8″ pots and grown in a controlled environment room. The growth conditions used are; 1) soil composition: 75% L&P fine-grade peat, 12% screened sterilized loam, 10% 6 mm screened, lime-free grit, 3% medium grade vermiculite, 3.5 kg Osmocote per m³ soil (slow-release fertilizer, 15-11-13 NPK plus micronutrients), 0.5 kg PG mix per m³ (14-16-18 NPK granular fertilizer plus micronutrients, 2) 16 h photoperiod (400W sodium lamps providing irradiance of ca. 750 μE s⁻¹ m⁻²), 18 to 20° C. day and 14 to 16° C. night temperature, 50 to 70% relative air humidity and 3) pest control: sulfur spray every 4 to 6 weeks and biological control of thrips using Amblyseius caliginosus (Novartis BCM Ltd, UK).

Two sources of primary explants are used; scutellar and inflorescence tissues. For scutella, early-medium milk stage grains containing immature translucent embryos are harvested and surface-sterilized in 70% ethanol for 5 min. and 0.5% hypochlorite solution for 15-30 min. For inflorescences, tillers containing 0.5-1.0 cm inflorescences are harvested by cutting below the inflorescence-bearing node (the second node of a tiller). The tillers are trimmed to approximately 8-10 cm length and surface-sterilized as above with the upper end sealed with Nescofilm (Bando Chemical Ind. Ltd, Japan).

Preparation of DNA

Under aseptic conditions, embryos of approximately 0.5-1.0 mm length are isolated and the embryo axis removed. Inflorescences are dissected from the tillers and cut into approximately 1 mm pieces. Thirty scutella or 1 mm inflorescence explants are placed in the center (18 mm target circle) of a 90 mm Petri dish containing MDO.5 or L7D2 culture medium. Embryos are placed with the embryo-axis side in contact with the medium exposing the scutellum to bombardment whereas inflorescence pieces are placed randomly. Cultures are incubated at 25±° C. in darkness for approximately 24 h before bombardment. After bombardment, explants from each bombarded plate are spread across three plates for callus induction.

The standard callus induction medium for scutellar tissues (MDO.5) consists of solidified (0.5% Agargel, Sigma A3301) modified MS medium supplemented with 9% sucrose, 10 mg l⁻¹ AgNO₃ and 0.5 mg l⁻¹ 2,4-D (Rasco-Gaunt et al., 1999). Inflorescence tissues are cultured on L7D2 which consists of solidified (0.5% Agargel) L3 medium supplemented with 9% maltose and 2 mg l⁻¹ 2,4-D (Rasco-Gaunt and Barcelo, 1999). The basal shoot induction medium, RZ contains L salts, vitamins and inositol, 3% w/v maltose, 0.1 mg l⁻¹ 2,4-D and 5 mg l⁻¹ zeatin (Rasco-Gaunt and Barcelo, 1999). Regenerated plantlets are maintained in RO medium with the same composition as RZ, but without 2,4-D and zeatin.

Submicron gold particles (0.6 μm Micron Gold, Bio-Rad) are coated with a plasmid containing the DNA construct following the protocol modified from the original Bio-Rad procedure (Barcelo and Lazzeri, 1995). The standard precipitation mixture consists of 1 mg of gold particles in 50 μl SDW, 50 μl of 2.5 M calcium chloride, 20 μl of 100 mM spermidine free base and 5 μl DNA (concentration 1 μg μ⁻¹). After combining the components, the mixture is vortexed and the supernatant discarded. The particles are then washed with 150 μl absolute ethanol and finally resuspended in 85 μl absolute ethanol. The DNA/gold ethanol solution is kept on ice to minimize ethanol evaporation. For each bombardment, 5 μl of DNA/gold ethanol solution (ca. 60 μg gold) is loaded onto the macrocarrier.

Transformation and Regeneration

Particle bombardments are carried out using DuPont PDS 1000/He gun with a target distance of 5.5 cm from the stopping plate at 650 psi acceleration pressure and 28 in. Hg chamber vacuum pressure.

For callus induction, bombarded explants are distributed over the surface of the medium in the original dish and two other dishes and cultured at 25±1° C. in darkness for three weeks. Development of somatic embryos from each callus are periodically recorded. For shoot induction, calluses are transferred to RZ medium and cultured under 12 h light (250 μE s⁻¹ m⁻², from cool white fluorescent tubes) at 25±1° C. for three weeks for two rounds. All plants regenerating from the same callus are noted. Plants growing more vigorously than the control cultures are potted in soil after 6-9 weeks in R0 medium. The plantlets are acclimatized in a propagator for 1-2 weeks. Thereafter, the plants are grown to maturity under growth conditions described above.

For callus induction, bombarded explants are distributed over the surface of the medium in the original dish and two other dishes and cultured at 25±1° C. in darkness for three weeks. Development of somatic embryos from each callus are periodically recorded. For shoot induction, calluses are transferred to RZ medium and cultured under 12 h light (250 μE s-⁻¹ m⁻², from cool white fluorescent tubes) at 25±1° C. for three weeks for two rounds. All plants regenerating from the same callus are noted. Plants growing more vigorously than the control cultures are potted in soil after 6-9 weeks in R0 medium. The plantlets are acclimatized in a propagator for 1-2 weeks. Thereafter, the plants are grown to maturity under growth conditions described above.

For callus induction, bombarded explants are distributed over the surface of the medium in the original dish and two other dishes and cultured at 25±1° C. in darkness for three weeks. Development of somatic embryos from each callus are periodically recorded. For shoot induction, calluses are transferred to RZ medium and cultured under 12 h light (250 μE s⁻¹ m⁻², from cool white fluorescent tubes) at 25±1° C. for three weeks for two rounds. All plants regenerating from the same callus are noted. Plants growing more vigorously than the control cultures are potted in soil after 6-9 weeks in R0 medium. The plantlets are acclimatized in a propagator for 1-2 weeks. Thereafter, the plants are grown to maturity under growth conditions described above.

### Example 3

### Method of Wide Hybridization Between Maize and Wheat

A wide hybridization cross is performed between a male donor maize plant and a female acceptor wheat plant. The maize plant has stably incorporated into its genome the transfer cassette described in Example 1, while the wheat plant has stably incorporated into its genome the target site of Example 1. In addition, the wheat plant genome also has stably incorporated an expression cassette comprising a nucleotide sequence encoding the appropriate recombinase, in this case FLP recombinase.

The wide cross is performed as follows:

Plants are grown at temperatures ranging between 15° C. to 27° C. with a photoperiod of about 16 to 8 hours. Several days before expected anthesis middle florets are removed and the remaining emasculated and isolated to prevent cross-pollination and desiccation. On the day anthesis is expected to occur, florets will be pollinated with freshly collected maize pollen. Two days after pollination, plants will be treated with growth regulators (2,4-D 100 mgl⁻¹, pH 5.5). Application will be performed either by dipping the florets in the solution or injecting it into the uppermost internode.

Embryos will be rescued 18 to 21 days after pollination. At this time they will have developed scutellum, and coleorhize. Brown, necrotic spots on scutellum will be the first signs of degeneration indicating that embryos are too old for culture. Grains (about 3 mm of length) will be isolated and sterilized by immersing in 70% ethanol followed by 3 minutes in 0.05% HgC1₂ and 15 minutes in 10% bleach (both with a drop of Tween) and thorough washing.

Isolated embryos are cultured in vitro. To promote germination scutellum should be placed directly on embryo culture medium and cultured in dark at 18° C. Germinating embryos will be transferred to light (12 hours, 120 μmol m⁻² s⁻¹). Various embryo culturing media may be used including, but not limited to, 190-2 (Zhuang et al. (1983) Cell and Tissue Culture Techniques for Cereal Crop Improvement 431 Science Press.) or MS supplemented with IAA 0.1 mgl⁻¹, kinetin 1 mgl⁻¹, sucrose 601 gl⁻¹ (Zhang et al. (1996) Euphytica 90:315-324). Finally, chromosome doubling of haploid plants is carried out using 0.1% colchicine supplemented with 2% DMSO.

As shown in described in Example 1, the acceptor target site is designed so that a successful recombination event activates the expression of the bar gene. The progeny from the wide cross will be sprayed with herbicide Basta to select for the site-specific recombination event. The same treatment should identify the wheat haploid seedlings containing a DNA fragment transferred from the main chromosomes.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method for targeting the insertion of a nucleotide sequence of interest into a target site within the genome of an acceptor plant, said method comprising:
a) sexually crossing a donor plant and the acceptor plant to produce a hybrid cell wherein,
i) the genome of the donor plant comprises at least one DNA construct comprising a transfer cassette comprising in series, a first recombination site, said nucleotide sequence of interest, and a second recombination site, wherein the first and second recombination sites are non-identical;
ii) the genome of said acceptor plant comprises the target site, said target site comprising in series, the first recombination site, a DNA sequence, and the second recombination site, wherein the first and the second recombination sites are non-identical and correspond to the non-identical sites of the transfer cassette; and,
iii) the acceptor plant and the donor plant are from different species;
b) providing a recombinase, or variant or fragment thereof, that implements recombination at the non-identical recombination sites such that the nucleotide sequence of interest is inserted at the target site;
c) recovering a transgenic haploid cell comprising the nucleotide sequence of interest, wherein the transgenic haploid cell is capable of generating a plant; and,
d) generating a haploid transgenic plant comprising the nucleotide sequence of interest integrated into the target site.

2. The method of claim 1, further comprising generating a diploid transgenic plant comprising the nucleotide sequence of interest.

3. The method of claim 1, wherein the donor plant and the acceptor plant are from the same family.

4. The method of claim 3, wherein the donor plant and the acceptor plant are from the family Poaceae.

5. The method of claim 4, wherein the donor plant and the acceptor plant are from different genera, said genera selected from the group consisting of Zea, Triticum, Hordeum, Sorghum, Oryza, and Avena.

6. The method of claim 5, wherein the donor plant is from the genus Zea and acceptor plant is from the genus Triticum.

7. The method of claim 1, wherein the donor plant and the acceptor plant are from the same genus.

8. The method of claim 7, wherein the genus is selected from the group consisting of Zea, Triticum, Hordeum, Sorghum, Oryza, and Avena.

9. The method of claim 1, wherein said non-identical recombination sites are selected from the group consisting of FRT, mutant FRT, LOX and mutant LOX sites.

10. The method of claim 9, wherein the recombinase is selected from the group consisting of FLP, Cre, and chimeric FLP/Cre.

11. The method of claim 9, wherein said sites are selected from the group consisting of FRT and mutant FRT sites.

12. The method of claim 11, wherein said mutant FRT sites are selected from the group consisting of FRT5, FRT6 and FRT7.

13. The method of claim 11, wherein said recombinase is FLP, or an active variant or fragment thereof.

14. The method of claim 1, wherein a nucleotide sequence encoding said recombinase is stably incorporated into the genome of said acceptor plant, and said nucleotide sequence encoding said recombinase is operably linked to a promoter.

15. The method of claim 1, wherein said target site further comprises a nucleotide sequence encoding said recombinase, said nucleotide sequence encoding said recombinase is operably linked to a promoter, and wherein the nucleotide sequence encoding the recombinase is located between the first and the second recombination sites.

16. The method of claim 1, wherein the target site further comprises a selectable marker located between the first and the second recombination sites.

17. The method of claim 1, wherein the DNA construct comprising the transfer cassette further comprises a selectable marker located between the first and the second recombination sites.

18. The method of claim 17, wherein the selectable marker is not operably linked to a promoter, and wherein the target site further comprises a promoter active in said plant, such that the promoter is operably linked to the first recombination site of the target site, whereby insertion of the selectable marker at the target site results in the selectable marker being operably linked to the promoter.

19. A method for the stable introduction of a nucleotide sequence of interest into the genome of a plant, said method comprising:
a) sexually crossing a donor plant and an acceptor plant to produce a hybrid cell wherein,
i) the genome of said donor plant comprises at least one DNA construct comprising a transfer cassette comprising in series, a first recombination site, an expression cassette comprising said nucleotide sequence of interest, and a second recombination site, wherein the first and the second recombination sites are non-identical,
ii) the genome of said acceptor plant comprises a target site comprising in series, the first recombination site, a DNA sequence, and the second recombination site, wherein the first and the second recombination sites are non-identical and correspond to the non-identical sites of the transfer cassette; and,
iii) the acceptor plant and the donor plant are from different species;
b) providing a recombinase or variant or fragment thereof, that implements recombination at the non-identical recombination sites such that the nucleotide sequence of interest is inserted at the target site;
c) recovering a transgenic haploid cell comprising the nucleotide sequence of interest, wherein said haploid cell is capable of generating a plant; and,
d) generating a haploid transgenic plant comprising the nucleotide sequence of interest is stably incorporated into its genome.

20. The method of claim 19, further comprising generating a diploid transgenic plant comprising the nucleotide sequence of interest.

21. The method of claim 19, wherein the donor plant and the acceptor plant are from the same family.

22. The method of claim 21, wherein the donor plant and the acceptor plant are from the family Poaceae.

23. The method of claim 22, wherein the donor plant and the acceptor plant are from different genera, said genera selected from the group consisting of Zea, Triticum, Hordeum, Sorghum, Oryza, and Avena.

24. The method of claim 23, wherein the donor plant is from the genus Zea and the acceptor plant is from the genus Triticum.

25. The method of claim 19, wherein the donor plant and the acceptor plant are from the same genus.

26. The method of claim 25, wherein the genus is selected from the group consisting of Zea, Triticum, Hordeum, Sorghum, Oryza, and Avena.

27. The method of claim 19, wherein the non-identical recombination sites are selected from the group consisting of FRT, mutant FRT, LOX, and mutant LOX sites.

28. The method of claim 27, wherein the recombinase is selected from the group consisting of FLP, Cre, and chimeric FLP/Cre.

29. The method of claim 27, wherein said sites are selected from the group consisting of FRT and mutant FRT sites.

30. The method of claim 29, wherein said mutant FRT sites are selected from the group consisting of FRT5, FRT6, and FRT7.

31. The method of claim 29, wherein said recombinase is FLP, or an active variant or fragment thereof.

32. The method of claim 19, wherein a nucleotide sequence encoding said recombinase is stably incorporated into the genome of the acceptor plant, and said nucleotide sequence encoding said recombinase is operably linked to a promoter.

33. The method of claim 19, wherein said target site further comprises a nucleotide sequence encoding said recombinase, said nucleotide sequence encoding said recombinase is operably linked to a promoter, and wherein the nucleotide sequence encoding the recombinase is located between the first and the second recombination sites.

34. The method of claim 19, wherein the target site further comprises a selectable marker located between the first and the second recombination sites.

35. The method of claim 19, wherein the DNA construct comprising the transfer cassette further comprises a selectable marker located between the first and the second recombination sites.

36. The method of claim 35, wherein the selectable marker is not operably linked to a promoter, and wherein the target site further comprises a promoter active in said plant, such that the promoter is operably linked to the first recombination site of the target site, whereby insertion of the selectable marker at the target site results in the selectable marker being operably linked to the promoter.


SEQUENCE LISTING (4 sequences)

SEQ ID NO: 1 - DNA, 69 bp, Artificial Sequence
  wild type FRT recombination site
ccatggctagcgaagttcctattccgaagttcctattctctagaaagtataggaacttcagatctcgag

SEQ ID NO: 2 - DNA, 69 bp, Artificial Sequence
  FRT5 recombination site
ccatggctagcgaagttcctattccgaagttcctattcttcaaaaggtataggaacttcagtactcgag

SEQ ID NO: 3 - DNA, 72 bp, Artificial Sequence
  FRT6 recombination site
ccatggctagcgaagttcctattccgaagttcctattcttcaaaaagtataggaacttcagacgtcctcgag

SEQ ID NO: 4 - DNA, 72 bp, Artificial Sequence
  FRT7 recombination site
ccatggctagcgaagttcctattccgaagttcctattcttcaataagtataggaacttcactagttctcgag